(19) United States
(12) Patent Application Publication
(10) Pub. No.: US 2017/0319194 A1
(43) Pub. Date: Nov. 9, 2017

(54) HIGH FRICTION FASTENING FLAT BRAID

(71) Applicant: TELEFLEX MEDICAL INCORPORATED, MORRISVILLE, NC (US)

(72) Inventors: CARSTEN R. MAYESKI, Sharon, MA (US); Christopher V. Dymond, Franklin, MA (US); Michael J. Christian, West Bridgewater, MA (US)

(21) Appl. No.: 15/590,575

(22) Filed: May 9, 2017

Related U.S. Application Data
(60) Provisional application No. 62/333,600, filed on May 9, 2016.

(51) Int. Cl. A61B 17/04; D04C 1/02; A61L 17/04; A61B 17/00

(52) U.S. Cl. CPC A61B 17/0401; D04C 1/02; A61B 2017/00858; A61B 17/04; A61L 17/04

(57) ABSTRACT

A suture having a contoured surface may include a plurality of first ends having a first diameter and a plurality of second ends having a second diameter. The first and second ends may be braided together, wherein the first diameter is different than the second diameter to form the contoured surface. The first ends and the second ends may braided into a flat braid. In some embodiments, the first ends may comprise a material having a greater coefficient of friction than the second end. A method of securing first and second suture segments is also provided.

### PRIORITY

This patent application claims priority to U.S. Provisional Patent Application No. 62/333,600, titled High Friction Fastening Flat Braid and having a filing date of May 9, 2016, the disclosure of which is hereby incorporated by reference in its entirety.

### FIELD OF THE DISCLOSURE

The present disclosure relates generally to a flat braided suture. More particularly, the present disclosure relates to a high friction flat braided suture having a contoured surface made of different sized ends.

### BACKGROUND

Ultra high molecular weight polyethylene (UHMWPE) sutures have shown favorable strength in surgical procedures but are exceptionally lubricious, which can cause increased complexity and complications. For example, UHMWPE sutures can often slip when tied into a knot and/or otherwise fixed to a bone anchor. Attempts to address this problem include adding frictional coatings to the UHMWPE sutures, which increases cost and manufacturing lead time. Surgeons have also attempted to tie the UHMWPE sutures into larger, more cumbersome knots to ensure sufficient friction between the tied segments of suture, which can cause complications and patient discomfort.

Accordingly, it is desirable to provide a suture with improved mechanical and/or frictional properties.

### SUMMARY OF THE DISCLOSURE

The foregoing needs are met, to a great extent, by the present disclosure, wherein in one aspect an apparatus is provided that in some embodiments.

In accordance with one embodiment, a suture having a contoured surface is provided. The suture may include a plurality of first ends having a first diameter and a plurality of second ends having a second diameter. The first and second ends may be braided together, wherein the first diameter is different than the second diameter to form the contoured surface.

In accordance with another embodiment, a suture having a contoured surface is provided. The suture may include a plurality of first ends having a first diameter and a plurality of second ends having a second diameter. The first and second ends may be braided into a flat braid, wherein the first diameter is different than the second diameter to form the contoured surface. The first diameter may be at least about two times greater than the second diameter to form the contoured surface. At least one of the first ends of the first segment may be configured to be received between adjacent first ends of the second segment, and the at least one of the first ends of first segment may be configured to frictionally engage the adjacent first ends of the second segment.

In accordance with yet another embodiment, a method of securing first and second suture segments is provided. The method may include overlaying a first flat braid of the first suture segment onto a second flat braid of the second suture segment. The method may also include receiving an end of the first flat braid between adjacent ends of the second flat braid. The method may further include frictionally engaging the end of the first flat braid with at least one of the adjacent ends of the second flat braid.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a partial side view illustrating an exemplary suture in accordance with an embodiment of the disclosure.

FIG. 2 is a first partial top view of an exemplary suture in accordance with the disclosure.

FIG. 3 is a second partial top view of an exemplary suture in accordance with the disclosure.

FIG. 4 is an enlarged, cross-sectional view of an exemplary suture in accordance with the disclosure.

FIG. 5 is an enlarged, cross-sectional view of an exemplary suture doubled back on itself as if tied in a knot.

### DETAILED DESCRIPTION

The various embodiments will now be described with reference to the drawing figures, in which like reference numerals refer to like parts throughout. Some embodiments of the present disclosure may provide a braided suture having an undulated and/or contoured surface. The suture may be formed by braiding one or more first ends having a first width or diameter and one or more second ends having a second width or diameter, the first diameter being different than the second diameter which forms the contoured surface. The first diameter may be at least about two times the size of the second diameter. In some embodiments, the first ends may be formed of one or more fibers of a first material (e.g., Polyethylene Terephthalate, PET), and the second ends may be formed of one or more fibers of a second material (e.g., Ultra High Molecular Weight Polyethylene, UHMWPE). The first ends may have a first linear density greater at least about two times (e.g., 2.5 times) a second linear density of the second ends. The suture of the present disclosure may therefore provide improved frictional properties, for example, allowing increased knot security with fewer throws. The suture would also allow for smaller knots, improved patient comfort, and/or reduced chances of a complication.

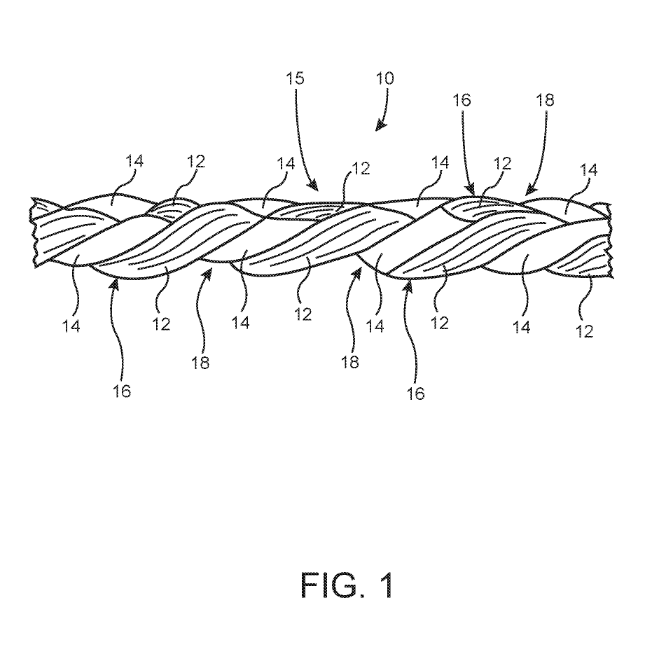

FIG. 1 illustrates a partial side view of a suture 10 according to some exemplary embodiments of the disclosure. The suture 10 may comprise a flat braid of one or more first strands or ends 12 and one or more second strands or ends 14. In some embodiments, at least one of the first ends 12 and/or the second ends 14 may include a plurality of fibers twisted and/or braided together. The first and second ends 12, 14 may have any number of cross-sections, such as circular, oval, square, and/or rectangular.

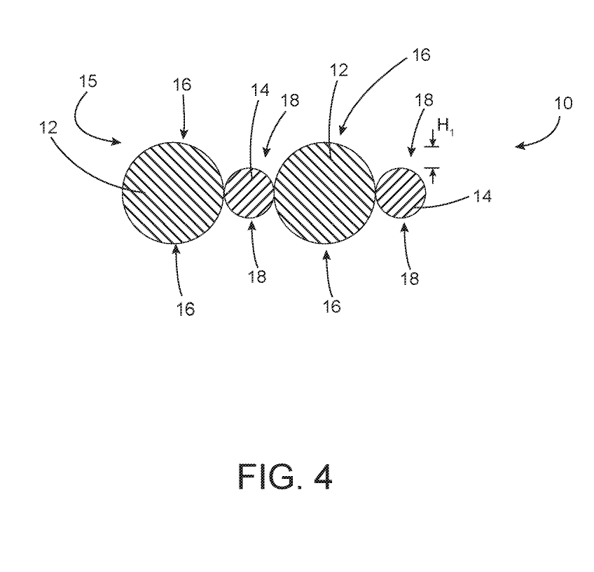

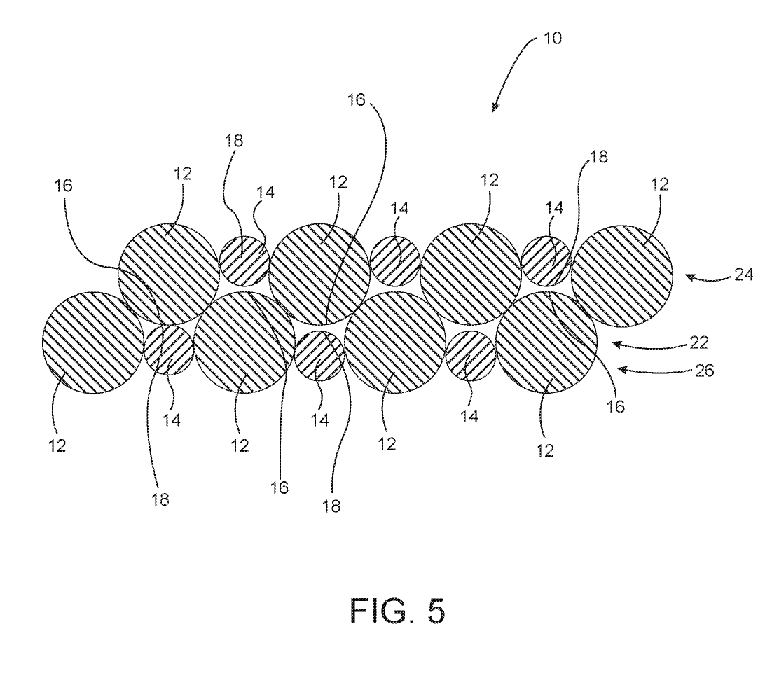

The flat braided configuration may be formed by a collapsed tubular configuration and/or a planar configuration with substantially laterally arranged ends 12, 14 (as depicted in FIGS. 4 and 5). In some embodiments, the flat braid may extend the entire length of the suture 10. In some embodiments, the flat braid may not extend the entire length of the suture 10. For example, the suture 10 may have one or more segments of any number of configurations such flat, square, round, hollow, and/or tubular. The first and second ends 12, 14 may continuously extend through the segments having the various configurations. In some embodiments, the suture 10 may alternate between one or more round and flat segments to form a round-flat-round (RFR) suture. For example, the flat segments of the RFR suture may be formed by introducing gaps into a round, tubular configuration, such that the suture would flatten into substantially flat, planar segments.

The first and second ends 12, 14 may comprise materials having different mechanical and/or frictional properties. For example, the first end 12 may comprise a first material such as PET, and the second end 14 may comprise a second material such as UHMWPE. The first material may have a greater coefficient of friction than the second material, and the second material may have a greater tensile strength than the first material. The first end 12 and/or the second end 14 may include one or more fibers that may be dyed and/or otherwise colored any color and/or left plain. In some embodiments, all, part, none, and/or of some of the fibers of the first end 12 and/or the second end 14 may be colored. In some embodiments, at least some of the fibers of the first end 12 may be colored green.

As further shown in FIG. 1, the first ends 12 may comprise a first diameter, and the second ends 14 may comprise a second diameter. The first diameter may be different than the second diameter to provide a variable width and/or contoured surface 15. In some embodiments, the first diameter may be at least about two times the second diameter. For example, the first diameter may be between about two and four times the second diameter. The contoured surface 15 may include one or more high portions 16 (shown by the shaded portions in FIG. 1) and one or more low portions 18 (shown as the unshaded portions 18 in FIG. 1). It may be appreciated that the first ends 12 may have substantially the same diameter (e.g., the first diameter) and/or substantially the same material (e.g., the first material), and the second end 14 may have substantially the same diameter (e.g., the second diameter) and/or substantially the same material (e.g., the second material). The first ends 12 and second ends 14 may be braided tightly so that there are no gaps in the suture 10 when the suture 10 is bent and/or twisted.

In some embodiments, the linear density of the first and second ends 12, 14 may be different, which may result in the first and second ends 12, 14 having different diameters. For example, the first ends 12 may have a linear density of at least about two times (e.g., about 2.5 times) greater than the linear density of the second ends 12. For example, in some embodiments, the first ends 12 may have a linear density of about 250 denier, and the second ends 14 may have a linear density of about 100 denier.

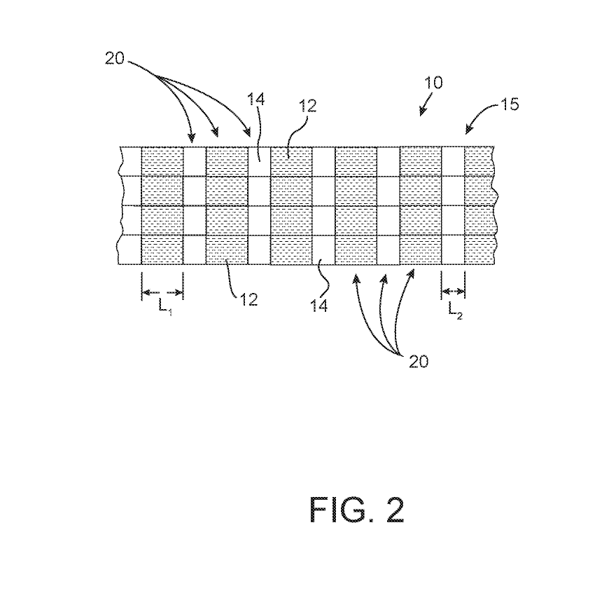

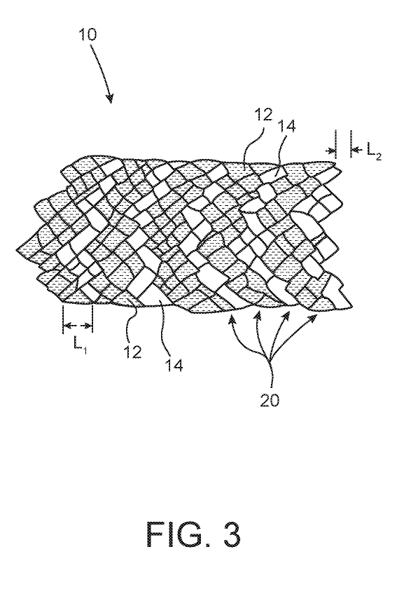

As further shown in the top views of FIGS. 2 and 3, the combination of the high portions 16 and the low portions 18 may form a stepped pattern 20. For example, the first ends 12 may include exposed segments that provide a pattern length L1, and the second ends 14 may include exposed segments that provide a pattern length L2. Due to the relative diameters of the first and second ends 12, 14 and/or the braiding of the suture 10, the pattern length L1 and the pattern length L2 may be unequal. For example, due to the first diameter being greater than the second diameter and/or the braiding of the suture 10, the pattern length L1 may be greater than the pattern length L2. In some embodiments, the pattern length L1 may be at least twice the length of pattern length L2. It should be understood that FIG. 2 depicts an idealized representation of the suture 10, and FIG. 3 is a more realistic drawing of the suture 10. For example, FIG. 2 depicts an idealized pattern 20 and lengths L1 and L2, and FIG. 3 depicts inconsistencies in the braided suture 10 resulting from the braiding process.

FIGS. 4 and 5 depict exemplary cross-sectional views of the suture 10. As depicted, the suture 10 may include a flat braid comprising the first ends 12 and the second ends 14 substantially laterally arranged. Due to the variable diameters of the suture 10, the contoured surface 15 may include recesses between the high portions 16 of the first ends 12. In some embodiments, the first ends 12 and the second ends 14 may alternate laterally to provide recesses between adjacent pairs of the first ends 12. The recesses may include a height H1 that may be defined by one half of the difference between the first diameter of the first end 12 and the second diameter of the second end 14. However, in some embodiments, the second ends 14 may be disposed vertically offset of the first ends 12, which may provide recesses with a greater height H1.

While FIG. 4 only depicts four combined ends 12, 14 and FIG. 5 depicts eight combined ends 12, 14, it should be understood that the suture 10 may include any number of the first and second ends 12, 14. For example, the suture 10 may include many more first and second ends 12, 14 than depicted in FIGS. 4 and 5. The number of the first ends 12 may be the same as or different than the number of the second ends 14. In some embodiments, the suture 10 may be about 1.5 to 2 mm wide. However, the suture 10 may have other width in accordance with this disclosure.

FIG. 5 illustrates an exemplary embodiment of the suture 10 having a first segment 24 and a second segment 26 in an interlocking and/or meshing configuration. As depicted, the suture 10 may provide a favorable frictional engagement when the first and second segments 24, 26 are overlaid and/or placed in contact with each other. The frictional engagement between the first and second segments 24, 26 may be performed when the suture is tied into a knot and/or otherwise doubled back on itself. During the engagement, at least one of the high portions 16 of the first segment 24 may be received in recesses between adjacent high portions 16 of the second segment 26 of the suture 10. In some embodiments, the first and second diameters may be sized such the at least one of the high portions 16 of the first segment 24 may frictionally engage two adjacent first ends 12 during meshing of the first and second segments 24, 26. As depicted, any number of the high portions 16 of the first segment 24 may engage the high portions 16 of the second segment 26 to provide more or less frictional engagement between the first and second segments 24, 26. For example, substantially the entire widths the first and second segments 24, 26 may overlap to provide a greater frictional engagement, or the first and second segments may be overlaid such that just the peripheral ends 12, 14 overlap. The fact that the first and second segments 24, 26 may mesh when the suture 10 is tied to a knot and/or doubled back on itself allows the suture 10 to have a relatively high friction and less lubricity making it better suited for being tied and/or otherwise secured. For example, the frictional engagement of the suture 10 is not relying on the lower coefficient of friction of the second end 14 (e.g., UHMWPE) for the frictional engagement required in knot-tying. Providing the first ends 12 with an increased coefficient of friction would further increase the frictional engagement between the first and second segments 24, 26. In some embodiments, the first and second segments 24, 26 may be of the same suture 10. In some embodiments the first segment 24 may be of a first suture 10, and the second segment 26 may be of a second suture 10.

It will be understood that the nature of meshing of the first and second segments 24, 26 are idealized in the example shown in FIGS. 4 and 5. In some situations, the meshing of the first and second segments 24, 26 may be accomplished in not such an idealized manner. For example, the first and second segments 24, 26 may be skewed with respect to each other, and therefore the first segment 24 does not completely overlap the second segment 26. However, the general principle of the high portions 16 frictionally engaging each other in a meshing or partial meshing manner may cause the suture 10 to have increased friction when doubled back on itself, bent, and/or tied into a knot. It should also be understood that more than the two exemplary segments 24, 26 of the suture 10 may frictionally engage each other. For example, any number of segments (e.g., two or more) may be overlaid upon each other to frictionally secure the suture 10. It should be further understood that the method may be applied to secure two or more different sutures 10 by frictionally engaging respective segments.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A suture having a contoured surface, the suture comprising:
a plurality of first ends having a first diameter; and
a plurality second ends braided with the first ends, the second ends having a second diameter,
wherein the first diameter is different than the second diameter to form the contoured surface.

2. The suture of claim 1, wherein the first ends and the second ends are braided into a flat braid.

3. The suture of claim 1, wherein the first ends comprise a first material, and the second ends comprises a second material, the first material having a greater coefficient of friction than the second material.

4. The suture of claim 3, wherein the first material comprises polyethylene terephthalate (PET).

5. The suture of claim 4, wherein the first ends comprise at least two fibers of PET twisted together.

6. The suture of claim 3, wherein the second material comprises ultra high molecular weight polyethylene (UHMWPE).

7. The suture of claim 6, wherein the second ends comprise at least two fibers of UHMWPE twisted tougher.

8. The suture of claim 1, wherein the first ends comprises a first linear density, and the second ends comprise a second linear density, the first linear density being greater than the second linear density.

9. The suture of claim 8, wherein the first linear density is at least about two times greater than the second linear density.

10. The suture of claim 1, wherein the first diameter is at least about two times greater than the second diameter.

11. The suture of claim 1, further comprising a first segment and a second segment, wherein the first segment is configured to mesh with the second segment.

12. The suture of claim 11, wherein at least one of the first ends of the first segment is configured to be received between adjacent first ends of the second segment.

13. The suture of claim 12, wherein the at least one of the first ends of the first segment is configured to frictionally engage the adjacent first ends of the second segment.

14. The suture of claim 1, wherein the first ends and the second ends alternate.

15. A suture having a contoured surface, the suture comprising:
a first segment and a second segment, the first and second segments comprising:
a plurality of first ends having a first diameter, and
a plurality of second ends braided with the first ends into a flat braid, the second ends having a second diameter,
wherein the first diameter is at least about two times greater than the second diameter to form the contoured surface, and
wherein at least one of the first ends of the first segment is configured to be received between adjacent first ends of the second segment, and the at least one of the first ends of first segment is configured to frictionally engage the adjacent first ends of the second segment.

16. The suture of claim 15, wherein the first ends comprise PET.

17. The suture of claim 15, wherein the second ends comprise UHMWPE.

18. A method of securing first and second suture segments, the method comprising:
overlaying a first flat braid of the first suture segment onto a second flat braid of the second suture segment;
receiving an end of the first flat braid between adjacent ends of the second flat braid; and
frictionally engaging the end of the first flat braid with at least one of the adjacent ends of the second flat braid.

19. The method of claim 18, comprising frictionally engaging the end of the first flat braid with both of the adjacent ends of the flat braid.

20. The method of claim 18, wherein the first and second suture segments are of a single suture.
